(19) United States
(12) Patent Application Publication
(10) Pub. No.: US 2023/0355671 A1
(43) Pub. Date: Nov. 9, 2023

(54) Upregulation of Cathelicidin Gene Expression as an Adjuvant to Other Treatments for Diseases

(71) Applicant: The Board of Trustees of the Leland Stanford Junior University, Stanford, CA (US)

(72) Inventors: Annelise E. Barron, Woodside, CA (US); John A. Fortkort, Austin, TX (US)

(73) Assignee: The Board of Trustees of the Leland Stanford Junior University, Stanford, CA (US)

(21) Appl. No.: 17/906,727

(22) PCT Filed: Mar. 18, 2021
(86) PCT No.: PCT/US2021/023032

§ 371 Date: Sep. 19, 2022

Related U.S. Application Data
(60) Provisional application No. 62/991,564, filed on Mar. 18, 2020.

(51) Int. Cl. A61K 35/17; A61K 35/768; A61K 45/06

(52) U.S. Cl. CPC A61K 35/17; A61K 35/768; A61K 45/06

(57) ABSTRACT

A method for treating a subject suffering from a disease is provided. The method includes diagnosing the subject as suffering from a disease; upregulating the cathelicidin gene CAMP in the subject; and administering to the subject at least one treatment for the disease.

### CROSS-REFERENCE TO RELATED APPLICATION

This application claims the benefit of priority of U.S. Patent Application No. 62/991,564, filed Mar. 18, 2020, having the same inventors and entitled “UPREGULATION OF CATHELICIDIN GENE EXPRESSION AS AN ADJUVANT TO OTHER TREATMENTS FOR DISEASES,” which is incorporated herein by reference in its entirety.

### FIELD OF THE DISCLOSURE

The present disclosure relates generally to methods for the treatment and prevention of diseases, and more particularly to methods for treating and preventing diseases by upregulating cathelicidin in a subject as an adjuvant to other therapies or treatments.

### BACKGROUND OF THE DISCLOSURE

The human immune system must contend with significant challenges posed by a variety of diseases and pathogens. These include the challenges posed by aggressive cancers, such as glioblastoma multiforme (GBM), and virulent pathogens, such as the coronavirus disease 2019 (COVID-19).

Natural killer (NK) cells are potent innate immune effector cells that perform critical roles in the body's natural immune system. These cells form an important line of defense against diseases and pathogens. In particular, NK cells kill cells that have become infected with viruses or other intracellular pathogens such as Mycobacterium tuberculosis bacteria, and also attack tumor cells. NK cells operate through a complex series of interactions that allow the human body to protect itself from diseases and pathogens. These interactions rely heavily on a class of proteins known as cytokines, which include interleukin 2 (IL-2) and interleukin 15 (IL-15).

IL-2 is a cytokine signaling molecule which regulates the activities of white blood cells (leukocytes, also called lymphocytes) that are responsible for immunity. IL-2 is thus part of the body's natural response to infection, and allows the body to discriminate between foreign (“non-self”) and native (“self”) entities. IL-2 mediates its effects by binding to IL-2 receptors, which are expressed by lymphocytes. The major sources of IL-2 are activated CD4+ T cells and activated CD8+ T cells. CD8+ T cells are antigen-specific and provide an enhanced protective response when the same antigen is encountered again in the future. Consequently, these cells are often referred to as CD8+ memory cells.

IL-15 shares many biological activities with IL-2. The protein encoded by IL-15 regulates T cell and NK cell activation and proliferation. IL-15 and IL-2 bind to common hematopoietin receptor subunits. Consequently, in some situations, IL-15 and IL-2 compete for the same receptor, and negatively regulate each other's activity. The number of CD8+ memory cells is shown to be controlled by a balance between IL-15 and IL-2.

Recently, NK cell-based immunotherapy has emerged as a promising therapeutic approach for treating solid tumors and hematological malignancies. For example, a recent study by Prins et al. showed (in a mouse model) that the administration of immature dendritic cells (DCs) induce dramatic and enhanced antitumor immune responses within the central nervous system (CNS). In particular, it was demonstrated that NK and CD4 cells act in concert to protect against melanoma tumor formation in the brain. See Prins et al., “NK and CD4 Cells Collaborate to Protect against Melanoma Tumor Formation in the Brain”, The Journal of Immunology Dec. 15, 2006, 177 (12) 8448-8455.

In another study, anti-tumor effects of activated human NK cells were demonstrated in orthotopic human brain tumor models. See Lee, Se Jeong et al. “Natural killer (NK) cells inhibit systemic metastasis of glioblastoma cells and have therapeutic effects against glioblastomas in the brain.” BMC cancer vol. 15 1011. 24 Dec. 2015, doi:10.1186/s12885-015-2034-y. Orthotopic models involve the implantation or seeding of tumor cell lines or patient-derived cell xenografts into the corresponding tissue in animal models. This strategy allows tumor development to be assessed in a relevant environment, and allows the efficacy of a treatment to be evaluated in a preclinical tumor model that mimics the disease process in humans. With orthotopic models, primary tumor growth can be closely monitored and accurately quantified, along with metastatic activity and response to treatment therapies. Glioblastoma, also known as glioblastoma multiforme (GBM), is an aggressive cancer that originates in the brain. It is the most commonly occurring type of malignant brain tumor. This type of aggressive tumor is associated with a high mortality rate and a high rate of reoccurrence.

In the study by Lee et al., human NK cells were engrafted in the brains of immunodeficient NSG (NOD (non-obese diabetic) scid (severe combined immune-deficient) gamma) mice. These mice lack mature T cells, B cells, and NK cells, are deficient in multiple cytokine signaling pathways, and have several defects in innate immunity. These immunodeficiencies permit the engraftment of a wide range of primary human cells, and thus allow modeling of human biology and disease. The engraftment was augmented with Interleukin 2 (IL-2) or Interleukin 15 (IL-15). It was found in this mouse model that NK cells exert significant anti-tumor effects against orthotopically implanted human GBM with no discernible toxicity.

Some studies have suggested that the effects of NK cell treatment may be improved if the treatment is combined with other therapies. For example, a study by Chen et al. found that the use of a particular type of NK cell (EGFR-CAR NK-92 cells) in combination with oncolytic herpes simplex virus 1 (oHSV-1) resulted in more efficient killing of MDA-MB-231 tumor cells, and significantly longer survival of tumor-bearing mice when compared to either therapy by itself. See Chen X, Han J, Chu J, Zhang L, Zhang J, Chen C, et al. A combinational therapy of EGFR-CAR NK cells and oncolytic herpes simplex virus 1 for breast cancer brain metastases. Oncotarget. (2016) 7:27764-77. 10.18632/oncotarget.8526.

### SUMMARY OF THE DISCLOSURE

In one aspect, a method is provided for treating a subject suffering from a disease. The method comprises diagnosing the subject as suffering from a disease; upregulating the cathelicidin gene CAMP in the subject; and administering to the subject at least one treatment for the disease.

In another aspect, a method is provided for treating a subject suffering from a disease. The method comprises diagnosing the subject as suffering from a disease; treating the disease; and upregulating the cathelicidin gene CAMP in the subject.

In a further aspect, a method is provided for treating a subject suffering from a disease. The method comprises diagnosing the subject as suffering from a disease; causing the subject to undergo an exercise therapy; and administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof.

In still another aspect, a method is provided for treating a subject suffering from a disease. The method comprises diagnosing the subject as suffering from a disease; causing the subject to undergo a therapy selected from the group consisting of (i) sauna therapy and/or (ii) hydrotherapy, wherein said therapy includes exposing the subject to a temperature of at least 74° C.; and administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof.

### BRIEF DESCRIPTION OF THE DRAWINGS

FIG. 1 is a flowchart illustrating some embodiments of a method for NK cell therapy in accordance with the teachings herein.

FIG. 2 is an illustration of various ways in which NK cells may be utilized in immunotherapy.

### DETAILED DESCRIPTION

Despite its promise, NK cell-based immunotherapy also has some notable limitations. For example, in some situations, NK cells are found to exhibit no cytotoxicity (that is, they exhibit no efficacy) against tumor cells. See, e.g., Minetto, Paola et al. “Harnessing NK Cells for Cancer Treatment.” Frontiers in Immunology vol. 10 2836. 6 Dec. 2019, doi:10.3389/fimmu.2019.02836. Without wishing to be bound by theory, this result is believed to arise from the action of Human Leukocyte Antigen (HLA)-I-specific inhibitory receptors within the tumor microenvironment.

In particular, NK cells distinguish between healthy autologous cells and unhealthy cells by ascertaining appropriate levels of expression of self-HLA alleles. Many tumor cells suffer a loss of HLA molecules and have an expression (or overexpression) of ligands for NK cell activating receptors. Thus, these tumor cells may be effectively recognized (and killed by) NK cells. Unfortunately, some tumor cells also maintain appropriate levels of expression of self-HLA alleles, and are thus indistinguishable from healthy cells on this basis. These tumor cells thus exhibit resistance to NK cell therapy. See Moretta L, Bottino C, Pende D, Vitale M, Mingari MC, Moretta A. Different checkpoints in human NK-cell activation. Trends Immunol. (2004) 25:670-6. doi: 10.1016/j.it.2004.09.008; and Pesce S, Greppi M, Grossi F, Del Zotto G, Moretta L, Sivori S, et al. PD/1-PD-Ls checkpoint: insight on the potential role of NK cells. Front Immunol. (2019) 10:1242. doi: 10.3389/fimmu.2019.01242.

It will be appreciated from the foregoing that further improvements in the art are needed for NK cell-based immunotherapy. In particular, a need exists in the art for improving the effectiveness of NK cell therapy and other treatments for diseases and infections. These and other needs may be addressed with the methodologies and compositions disclosed herein.

It has now been found that some or all of the foregoing needs may be met with the systems and methodologies disclosed herein. In a preferred embodiment, the method includes treating a subject through the upregulation of the cathelicidin gene CAMP in the subject as an adjuvant to other therapies that may be utilized to treat various diseases and maladies. For example, in one preferred embodiment, upregulation of the cathelicidin gene CAMP in a subject may be utilized as an adjuvant to Natural Killer (NK) cell therapy as, for example, by performing the upregulation before, during or after infusion of NK white blood cells into the body of the subject. In some variations of this embodiment (which are especially preferred for the treatment of tumors), the NK cell therapy may be utilized in conjunction with an oncolytic virus treatment. These infused NK cells may be obtained, for instance, from umbilical cord blood (from an unrelated human donor), or from an irradiated NK cell line such as NK92.

As a further example, upregulation of the cathelicidin gene CAMP in a subject may be utilized as an adjuvant to an oncolytic virus treatment (alone or in combination with NK cell therapy) as, for example, by performing the upregulation before, during or after the oncolytic virus treatment. In still other embodiments, upregulation of the cathelicidin gene CAMP in a subject may be utilized as an adjuvant to an antimicrobial or antiviral treatment (alone or in combination with NK cell therapy) as, for example, by performing the upregulation before, during or after the antimicrobial or antiviral treatment. The antimicrobial or antiviral treatment may feature the use, for example, of therapeutic aminoglycosides, peptides, peptoids, collectins, pulmonary surfactants, or other suitable, pharmaceutically active compositions.

Without wishing to be bound by theory, the upregulation of the cathelicidin gene CAMP in the subject as an adjuvant to other therapies is believed to improve such therapies by enhancing the body's native immune system. As a result, the body's ability to tolerate, leverage or respond positively to various other therapies may be augmented, and improved or synergistic effects with such other therapies may be realized.

For example, upregulation of the cathelicidin gene CAMP in a subject as an adjuvant to NK cell therapy is believed to significantly enhance the efficacy of the NK cell therapy, especially in the treatment of tumors. The resulting augmented treatment is desirable in that NK cell therapy, as well as the augmented treatment, is inherently less expensive than other treatment options available to cancer patients (such as CAR T-cell therapy). Moreover, the augmented NK cell therapy is conducive to outpatient administration, and is not characterized by common CAR T-cell side effects such as Cytokine Release Syndrome, neuroinflammation and a significant mortality rate. In addition, the inherent safety of this approach allows the upregulation of the cathelicidin gene CAMP in the subject to be continued indefinitely after NK T-cell therapy which, in addition to the greater efficacy of the NK T-cell therapy as promoted by upregulation of the cathelicidin gene CAMP in the subject before or during NK cell therapy, may help to prevent relapse. This will be true, whether this is an anti-infective, or an anti-cancer therapy, since the product of the CAMP gene, the cathelicidin LL-37, is one of the bodies most effective host defense peptides, and is antiviral, antibacterial, antifungal, antiparasitic, and anticancer. See: Vandamme et al., “A comprehensive summary of LL-37, the factotum human cathelicidin peptide”, Cellular Immunology 280 (2012) 22-35.

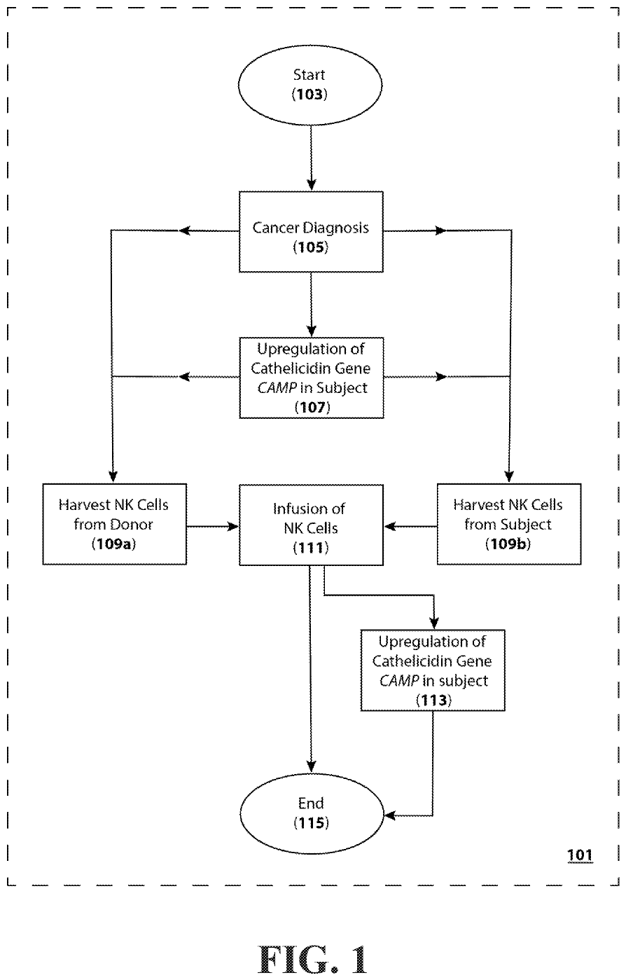

FIG. 1 depicts a first particular, non-limiting embodiment of a method in accordance with the teachings herein for treating a cancer patient. As seen therein, the method 101 commences 103 with a cancer diagnosis 105. The cancer diagnosis will typically be made by an oncologist or other suitable healthcare provider. A first suitable upregulating means is then employed to upregulate the cathelicidin gene CAMP in the subject 107, although in some embodiments, this step may be omitted. NK cells may then be harvested from a donor 109a or harvested from the subject 109b. After suitable treatment (such as, for example, expansion, treatment with cytokines, irradiation, and/or genetic modification), the harvested NK cells may be infused 111 into the subject. The process may end 115 here although, in some embodiments, a second suitable upregulating means (which may be the same as, or different from, the first upregulating means) is then employed to upregulate the cathelicidin gene CAMP in the subject 113. It will thus be appreciated that embodiments are possible which employ a first upregulating means only, a second upregulating means only, or both a first and second upregulating means. It will further be appreciated that upregulation of the cathelicidin gene CAMP expression in the subject may occur before, during and/or after NK cell therapy.

It will also be appreciated that, although cathelicidin induction is frequently disclosed herein as being an adjunct to NK cell therapy (and in particular, as a useful therapeutic step to be applied before, after, or simultaneously with NK cell therapy), more generally, cathelicidin induction may be an advantageous adjunct to various cancer therapies. These include, without limitation, CAR T cell therapy, radiation therapy, and chemotherapy.

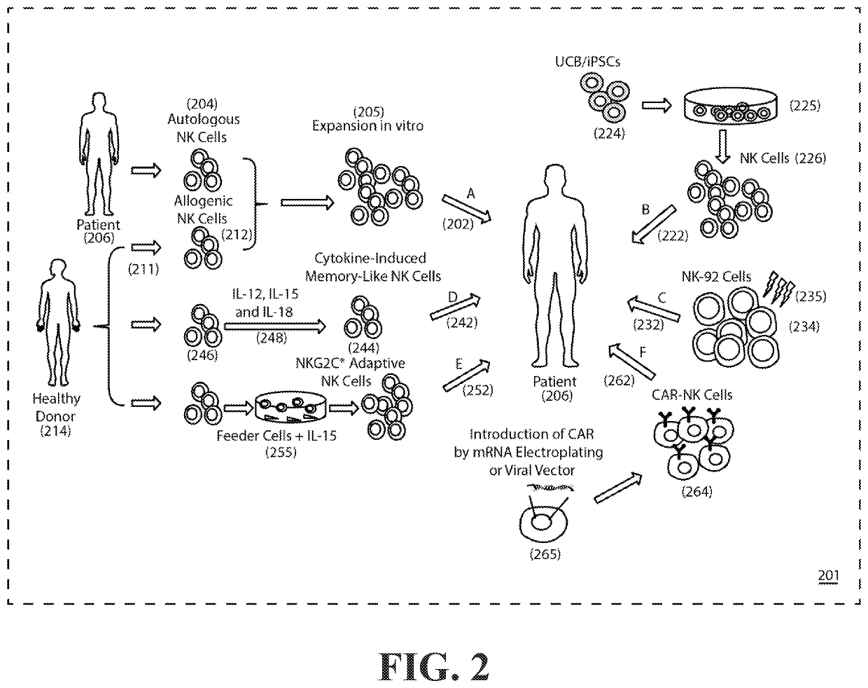

FIG. 2 illustrates some particular, non-limiting means 201 for sourcing or preparing NK cells that may be utilized in the systems and methodologies disclosed herein. In some embodiments, two or more of these methodologies may be utilized together. Similarly, in some embodiments, NK cells prepared by two or more of these methodologies may be administered to a patient, either separately or as a mixture.

In Scheme A 202, autologous NK cells 204 are harvested from a patient 206. The harvested cells are purified from peripheral blood (PB) and then expanded in vitro 205 through activation with suitable cytokines (such as, for example, IL-2 or IL-15). The expanded NK cells are then administered to the patient 206. In a variation of this method 211, allogeneic NK cells 212 with mismatched killer cell immunoglobulin-like receptors (KIRs) are harvested from a healthy donor 214, purified from PB and then activated with suitable cytokines 216 (such as, for example, IL-2 or IL-15) before administration into the patient 206. Of course, further variations of this embodiment are possible in which some NK cells 212 are harvested from the patient 206, and some are harvested from one or more donors 214. In these latter embodiments, the NK cells 212 may be administered to the patient separately or as a mixture.

In Scheme B 222, umbilical cord blood (UCB) and/or induced pluripotent stem cells (iPSCs) 224 are used as a source of functional NK cells 226. These NK cells may be co-cultured 225 with supportive feeder cells, or stimulated alone with a combination of cytokines, prior to administration to the patient. In some embodiments, this approach may utilize human iPSCs to produce NK cells with novel CARs that specifically target cancer cells in an antigen-specific manner.

In Scheme C 232, NK-92 cells 234 from the NK cell line are obtained. These cells are then irradiated 235 (for example, with 1000 cGy) prior to infusion. The use of NK-92 cells 234 in this approach is advantageous in that they display a robust and broad-spectrum cytotoxicity against malignant cells. Moreover, NK-92 cells are readily expanded under good manufacturing practice (GMP) conditions compared with allogeneic or UCB-derived NK cells. In addition, NK-92 cells may be efficiently manipulated with viral or non-viral vectors to enhance their targeting, homing, and killing activity. Finally, the safety of infusion with NK-92 cells has been confirmed in clinical trials.

In Scheme D 242, cytokine-induced memory-like NK cells 244 are obtained through pre-activation of human PB-derived NK cells 246 with one or more cytokines 248. Suitable cytokines may include, but are not limited to, IL-12, IL-15, and IL-18 and combinations thereof. The resulting cytokine-induced memory-like NK cells 244 are then administered to the patient.

In Scheme E 252, NKG2C+-adaptive NK cells 254 are preferentially expanded ex vivo from healthy donors. This may be achieved, for example, through culturing 255 with HLA-E-transfected 721.221 cells as feeder cells and a suitable cytokine such as, for example, IL-15. The resulting NKG2C+-adaptive NK cells 254 are then administered to the patient 206.

In Scheme F 262, NK cells are genetically modified with Chimeric Antigen Receptors (CARs) 264. This may occur, for example, through mRNA electroporation or viral vectors 265 in order to redirect the specificity and enhance the antitumor efficacy of the NK cell-based immunotherapy. The resulting CAR NK cells 264 are then administered to the patient 206.

It will be appreciated that, although cathelicidin induction is frequently disclosed herein as being an adjunct to NK cell therapy (and in particular, as a useful therapeutic step to be applied before, after, or simultaneously with NK cell therapy), more generally, cathelicidin induction may be an advantageous adjunct to various other cancer therapies. These include, without limitation, CAR T cell therapy, radiation therapy, chemotherapy, and oncolytic virus treatments.

The methodologies disclosed herein may also be utilized as an adjuvant to treatments for diseases caused by a variety of pathogens. These treatments may utilize various pharmaceutically active or effective materials such as, for example, pulmonary lung surfactants, collectins, peptides, peptoids, peptidomimetics, aminoglycoside antibiotics, or vaccines. The pathogens treatable with these therapies may include viruses (including, but not limited to, SARS-CoV-2), bacteria (including gram-positive and gram-negative bacteria), fungi, and parasites.

Upregulation of the cathelicidin gene CAMP may be utilized as an adjuvant to various diseases of various etymologies in accordance with the teachings herein. Examples of such diseases of a fungal etymology include, but are not limited to, aspergillosis; candidiasis; mucormycosis; histoplasmosis; blastomycosis; coccidioidomycosis; and paracoccidioidomycosis. Examples of such diseases of a bacterial etymology include, but are not limited to, brucellosis; campylobacter infections; cat-scratch disease; chlamydial infections; cholera; Escherichia coli infections; gonorrhea; klebsiella, enterobacter, and serratia infections; legionella infections; meningococcal infections; pertussis, plague, Mycobacterium tuberculosis infections, pseudomonas infections; salmonella infections; shigellosis; typhoid fever; and tularemia; anthrax; diphtheria; enterococcal infections; erysipelothricosis; listeriosis; nocardiosis; pneumococcal infections; staphylococcal infections; streptococcal infections; spirochete infections such as Borrelia Burgdorferri, bejel, yaws, and pinta; leptospirosis; Lyme disease; rat-bite fever; relapsing fever; syphilis; actinomycosis; bacteroides; botulism; clostridial infections; and tetanus. Examples of such diseases of a viral etymology include, but are not limited to, coronavirus (including COVID-19), enterovirus infections; bornavirus infections, herpesvirus infections; cytomegaloviruses such as HHV6A and HHV7, hepatitis A; hepatitis B; hepatitis C, Epstein-Barr virus, human papillomavirus (HPV); influenza; Japanese encephalitis (inflammation of the brain); measles, mumps, and rubella; polio; rabies; rotavirus; varicella; shingles (herpes zoster); and yellow fever), and HIV-1. Parasitic infections may include those involving Toxoplasma gondii and Trypanosoma cruzi.

As previously noted, in some embodiments of the systems and methodologies disclosed herein, upregulation of the cathelicidin gene CAMP in the subject may occur before, during and/or after the therapy to which it is an adjuvant. For example, if this therapy is NK cell therapy, a first method of upregulating the cathelicidin gene CAMP in the subject may be utilized before NK cell therapy, and a second method of upregulating the cathelicidin gene CAMP in the subject may be utilized after NK cell therapy. The first and second methods may be the same or different. By way of example but not limitation, in some embodiments, the second method may involve upregulating the cathelicidin gene CAMP in the subject at a lower level than the first method. In other embodiments, the second method may involve upregulating the cathelicidin gene CAMP in the subject in a different manner than that employed in the first method, or through the use of different materials.

As an example of the latter embodiment (and keeping with the example of NK cell therapy), a first pharmaceutical composition may be utilized to upregulate the cathelicidin gene CAMP in the subject prior to NK cell therapy, and a second pharmaceutical composition, which is distinct from the first pharmaceutical composition, may be utilized to upregulate the cathelicidin gene CAMP in the subject after NK cell therapy. The second pharmaceutical composition may differ from the first pharmaceutical composition in that, for example, the second pharmaceutical composition may upregulate the cathelicidin gene CAMP in the subject more weakly than the first pharmaceutical composition, or may be less cytotoxic than the first pharmaceutical composition. It will be appreciated that, in some embodiments, the upregulation of at least one protein product (which is preferably selected from the group consisting of hCAP-18 and LL-37 cathelicidin peptide) of CAMP gene in a subject may be verified before and/or after NK cell therapy, and this process may involve determining the levels or concentrations (relative or absolute) of these protein products in the blood of the patient or in the components thereof (here, it is noted that determining the concentrations or levels of these protein products in NK cells is especially preferred, since the concentrations or levels of these protein products in the blood serum of the patient may not be significantly or directly affected by the NK cell therapy).

Various means may be utilized in accordance with the teachings herein to upregulate the cathelicidin gene CAMP in a subject. These include, without limitation, the application to the subject of one or more pharmaceutical compositions of the type disclosed in U.S. Ser. No. 16/038,158, now published as U.S. 2019/0015361 (Barron et al.), “POLYTHERAPY MODULATING CATHELICIDIN GENE EXPRESSION MODULATION FOR THE TREATMENT OF ALZHEIMER'S DISEASE AND OTHER CONDITIONS”, which is incorporated herein by reference in its entirety, or the application to the subject of at least one substance selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof. One skilled in the art will appreciate that varying amounts of the foregoing compositions (or of various combinations of the foregoing compositions) may be required to achieve the desired level of upregulation of the cathelicidin gene CAMP in a particular subject or application, with the desired tolerability and minimal negative side effects.

Various other means may be utilized in accordance with the teachings herein to upregulate the cathelicidin gene CAMP in a subject. These include, without limitation, requiring the subject to undertake an exercise regime or to undergo radiation or light therapy, by vaccinating the subject with certain live vaccines (such as, for example, the bacille Calmette-Guerin (BCG) vaccine, or an oral poliovirus vaccine (OPV), which are known to provide upregulation of cathelicidin gene expression in human beings), or by causing the subject to undergo a therapy selected from the group consisting of (i) sauna therapy and (ii) hydrotherapy, wherein said therapy includes exposing the subject to a temperature of at least 74° C.

As previously noted, the NK cells utilized in some embodiments of the systems and methodologies disclosed herein may be obtained from various sources. For example, the NK cells may be obtained from the body of the subject to which they will be administered, or they may be obtained from one or more donors. Preferably, however, the NK cells are harvested from umbilical cord blood.

As previously noted, in some embodiments of the systems and methodologies disclosed herein, the NK cells may be genetically modified prior to being administered to a subject. More specifically, in some embodiments of the methodologies disclosed herein, CAMP gene expression may be upregulated in a donor. White blood cells (including NK cells) may be collected from that donor, and these white blood cells may be genetically transformed or modified through CAR (Chimeric Antigen Receptor) NK cell therapy (this therapy typically involves the addition of the gene for a special receptor that binds to a certain protein on the patient's cancer cells) to cause the NK cells to recognize or be receptive to particular markers or cellular ligands that may be present on cancer cells. Such markers may include, for example, PD-1 or PD-L1 (PD-1 is a protein found on T cells that regulates the body's immune responses in that, when it is bound to PD-L1 (another protein), it helps keep T cells from killing other cells, including cancer cells). The subject may then be treated using the methodologies disclosed herein for upregulating CAMP gene expression, after which the modified NK cells may be re-infused into the subject (here, it is noted that upregulated endogenous LL-37 expression will preferably have activated the subject's dendritic cells in such a way that NK cell therapy may work better). In other words, in this particular embodiment, if the donor is the subject, then the subject is treated to upregulated CAMP gene both before and after the steps of removing NK cells and then re-infusing transgenic NK cells.

The CAR NK cell therapy accomplishes two objectives. First of all, the NK cells are genetically altered so that they are receptive to the particular markers (e.g., PD-1 or PD-L1) found on cancer cells. Secondly, the NK cells are propagated in culture so there is a greater number of them. Hence, the success of the therapy is premised on the NK cells being focused on killing cancer cells in a subject, and on there being more of them.

In some embodiments, the methodologies disclosed herein may feature the treatment of a subject to upregulate the CAMP gene as an adjuvant to treatment of the subject with a pharmaceutical composition. In some embodiments, such a pharmaceutical composition may comprise at least one peptoid. Suitable peptoids for use in such embodiments may include, for example, the peptoids described in U.S. Pat. No. 8,445,632 (Barron et al.), entitled “Selective Poly-N-Substituted Glycine Antibiotics”, which is incorporated herein by reference in its entirety. It is also to be noted that halogenated analogs to any of the peptoids disclosed in the '632 patent may be produced in accordance with the teachings herein. Various cyclic peptoids may also be utilized in such embodiments including, but are not limited to, the peptoids disclosed in U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.), all of which are incorporated herein by reference in their entirety, or halogenated analogs of any of the foregoing.

Various halogenated peptoids and halogenated oligomers of N-substituted glycines may also be utilized in the methodologies disclosed herein. These include, without limitation, various halogenated analogs of the foregoing peptoids and oligomers of N-substituted glycines. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. However, the use of one or two bromo- or chloro-substitutions is preferred in many applications. Moreover, while the peptoids described herein may be halogenated at various locations, para-halogenation on the peptoids containing aryl rings is especially preferred in many applications, although ortho- and meta-substitution, or even perhalogentation, may be useful in some applications.

The peptoid compositions utilized in the compositions and methodologies described herein may also be alkylated, and preferably have terminal alkylation. Here, alkylation (and especially terminal alkylation) with a C10 or C13 tail is especially preferred. It has been found that such terminal alkylation can dramatically enhance the antibacterial activity of a peptoid, and in some cases, may cause a peptoid which otherwise has low antibacterial activity to have significant antibacterial activity.

In some embodiments of the methodologies disclosed herein, upregulation of the cathelicidin gene CAMP in a subject may be utilized to modify a treatment that such upregulation is an adjuvant to. For example, some antiviral or antibacterial compositions (such as, for example, peptoids or peptidomimetics) may exhibit unacceptable levels of cytotoxicity when used alone at pharmaceutically effective dosages or concentrations to treat an infection or disease. However, upregulation of the cathelicidin gene CAMP in a subject prior to, during and/or after administration of such a composition to the subject may allow such a composition to remain pharmaceutically effective while being applied to the subject at a lower dose or concentration. This may thus reduce the cytotoxicity of the composition to an acceptable level, thus increasing the number of drugs that may be safely administered to a subject.

In some embodiments, pharmaceutical compositions may be provided in accordance with the teachings herein which have a first component that upregulates the cathelicidin gene CAMP in the subject, and which have a second component that treats a specific pathogen or disease. For example, the first component may include at least one substance selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof. The second component may include, for example, a pharmaceutically active substance which is effective against at least one pathogen, or which may be used to treat at least one disease. Such pharmaceutically active substance may include, for example, one or more peptides, peptoids, peptidomimetics, aminoglycoside antibiotics, viral vectors, messenger RNA (mRNA), or other drugs.

It will thus be appreciated that the compositions and methodologies disclosed herein may be used in conjunction with various treatments and vaccines including, for example, MRNA vaccines and viral vector vaccines. These include, for example, the COVID-19 vaccines commercially available from Moderna and Pfizer-BioNTech, and the Janssen COVID-19 vaccine commercially available from Johnson & Johnson. In multidose treatments and vaccines, the cathelicidin gene CAMP in the subject may be upregulated before, during, and/or after the application of any dose.

Various time intervals (before or after) may be utilized between upregulation of the cathelicidin gene CAMP in a subject and the administration of another treatment in accordance with the teachings herein, and the time interval may be selected based on the particulars of the other treatment. However, this time interval will typically be no more than 24 hours, preferably less than 12 hours, more preferably less than 6 hours, and most preferably less than 3 hours.

The pharmaceutical compositions disclosed herein may be applied in various manners when applied contemporaneously with another treatment. For example, in some embodiments, a first pharmaceutical composition may be applied to a patient which upregulates the cathelicidin gene CAMP in a subject, and a second pharmaceutical composition (or compositions) may be applied which addresses the disease or condition the subject has been diagnosed with. In other embodiments, a single pharmaceutical composition may be administered to the subject which contains suitable ingredients to both upregulate the cathelicidin gene CAMP in the subject and address the disease or condition the subject has been diagnosed with.

The pharmaceutical compositions disclosed herein may be applied by various means, with consideration being given, for example, to the disease, condition or pathogen to be treated, the affected area or areas of the subject's body, or the condition or health of the subject. Such application may be topically, orally, intravenously, transdermally, subcutaneously, via inhalation, via infusion, or by other suitable means. The pharmaceutical compositions disclosed herein may be formulated into lozenges, solutions, liquids, gels, pastes, patches, powders, or other suitable forms.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

What is claimed is:

1. A method for treating a subject suffering from a disease, comprising:
diagnosing the subject as suffering from a disease;
upregulating the cathelicidin gene CAMP in the subject; and
administering to the subject at least one treatment for the disease.

2. The method of claim 1, wherein the at least one treatment is selected from the group consisting of Natural Killer (NK) cell therapy and oncolytic virus treatment.

3. The method of claim 1, wherein the at least one treatment includes NK cell therapy, and further comprising:
obtaining NK cells from umbilical cord blood.

4. The method of claim 1, further comprising:
verifying the upregulation of the cathelicidin gene CAMP in the subject.

5. The method of claim 1, further comprising:
verifying the upregulation of at least one protein product of CAMP gene in the subject, wherein said at least one protein product is selected from the group consisting of hCAP-18 and LL-37 cathelicidin peptide.

6. The method of claim 5, wherein the upregulation of cathelicidin LL-37 is verified in the subject by (a) measuring the concentration of LL-37 in a first biological sample obtained from the subject prior to upregulating cathelicidin in the subject, (b) measuring the concentration of LL-37 in a second biological sample obtained from the subject after upregulating cathelicidin in the subject; and (c) comparing the concentration of cathelicidin in the first and second samples.

7. The method of claim 6, wherein upregulation of LL-37 is verified in the subject only if the concentration of cathelicidin in the second sample is at least 3 times the concentration of cathelicidin in the first sample.

8. The method of claim 6, wherein upregulation of LL-37 is verified in the subject only if the concentration of cathelicidin in the second sample is at least 4 times the concentration of cathelicidin in the first sample.

9. The method of claim 6, wherein upregulation of LL-37 is verified in the subject only if the concentration of cathelicidin in the second sample is at least 5 times the concentration of cathelicidin in the first sample.

10. The method of claim 1, further comprising:
verifying the upregulation by (a) obtaining a sample of blood from the subject, and (b) measuring the concentration of LL-37 in the blood sample.

11. The method of claim 1, further comprising:
verifying the upregulation by (a) obtaining peripheral monocytes from the subject, and (b) measuring the levels of CAMP gene mRNA, or the concentration of LL-37 in the obtained peripheral monocytes.

12. The method of claim 2, further comprising:
modifying the NK cells such that they target a specific surface marker present on a pathogen causing the disease, thereby obtaining modified NK cells;
wherein infusing NK cells into the body of the subject includes infusing the modified NK cells into the body of the subject.

13. The method of claim 1, wherein upregulating cathelicidin in the subject includes treating the subject with a vaccine that upregulates cathelicidin.

14. The method of claim 1, wherein upregulating cathelicidin in the subject includes administering a pharmaceutical composition to the subject that upregulates cathelicidin.

15. The method of claim 14, wherein the pharmaceutical composition includes at least one substance selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof.

16. The method of claim 14, wherein the pharmaceutical composition includes at least two substances selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof.

17. The method of claim 14, wherein the pharmaceutical composition includes at least three substances selected from the group consisting of butyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof.

18. The method of claim 14, wherein the pharmaceutical composition includes at least four substances selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof.

19. The method of claim 1, wherein upregulating cathelicidin in the subject includes administering to the subject a pharmaceutical composition comprising the bacille Calmette-Guerin (BCG) vaccine.

20. The method of claim 1, wherein upregulating cathelicidin in the subject includes requiring the subject to undertake an exercise regime.

21. The method of claim 2, wherein upregulating the cathelicidin gene CAMP in the subject occurs prior to infusing the NK cells into the body of the subject.

22. The method of claim 2, wherein upregulating the cathelicidin gene CAMP in the subject occurs subsequent to infusing the NK cells into the body of the subject.

23. The method of claim 2, wherein upregulating the cathelicidin gene CAMP in the subject occurs both prior to and subsequent to infusing the NK cells into the body of the subject.

24. A method for treating a subject suffering from a disease, comprising:
diagnosing the subject as suffering from a disease;
treating the disease; and
upregulating the cathelicidin gene CAMP in the subject.

25. The method of claim 24, wherein upregulating the cathelicidin gene CAMP in the subject occurs prior to treating the disease.

26. The method of claim 24, wherein upregulating the cathelicidin gene CAMP in the subject occurs subsequent to treating the disease.

27. The method of claim 24, wherein upregulating the cathelicidin gene CAMP in the subject occurs both prior to and subsequent to treating the disease.

28. A method for treating a subject suffering from a disease, comprising:
diagnosing the subject as suffering from a disease;
causing the subject to undergo (a) an exercise therapy; and
administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, genistein, docosahexaenoic acid, caprylic acid, capric acid, lauric acid, and pharmaceutically acceptable salts thereof.

29. The method of claim 28, wherein the pharmaceutical composition upregulates the cathelicidin gene CAMP in the subject.

30. The method of claim 28, wherein administering the pharmaceutical composition to the subject includes prescribing the pharmaceutical composition to the subject.

31. The method of claim 28, further comprising:
treating the disease.

32. The method of claim 31, wherein administering a pharmaceutical composition to the subject occurs before the step of treating the disease.

33. The method of claim 31, wherein administering a pharmaceutical composition to the subject occurs after the step of treating the disease.

34. The method of claim 31, wherein administering a pharmaceutical composition to the subject occurs both before and after the step of treating the disease.

35. The method of claim 31, wherein causing the subject to undergo an exercise therapy occurs before the step of treating the disease.

36. The method of claim 31, wherein causing the subject to undergo an exercise therapy occurs after the step of treating the disease.

37. The method of claim 31, wherein causing the subject to undergo an exercise therapy occurs both before and after the step of treating the disease.

38. A method for treating a subject suffering from a disease, comprising:
diagnosing the subject as suffering from a disease;
causing the subject to undergo a therapy selected from the group consisting of (i) sauna therapy and (ii) hydrotherapy, wherein said therapy includes exposing the subject to a temperature of at least 74° C.; and
administering a pharmaceutical composition to the subject, wherein the pharmaceutical composition includes at least two substances selected from the group consisting of butyrate, phenylbutyrate, bexarotene, curcumin, resveratrol, retinol, beta-carotene, cholecalciferol, entinostat, docosahexaenoic acid, caprylic acid, capric acid, lauric acid, genistein and pharmaceutically acceptable salts thereof.

39. The method of claim 38, wherein the pharmaceutical composition upregulates the cathelicidin gene CAMP in the subject.

40. The method of claim 38, wherein administering the pharmaceutical composition to the subject includes prescribing the pharmaceutical composition to the subject.

41. The method of claim 38, further comprising:
treating the disease.

42. The method of claim 41, wherein administering the pharmaceutical composition to the subject occurs before the step of treating the disease.

43. The method of claim 41, wherein administering the pharmaceutical composition to the subject occurs after the step of treating the disease.

44. The method of claim 41, wherein administering the pharmaceutical composition to the subject occurs both before and after the step of treating the disease.

45. The method of claim 41, wherein causing the subject to undergo the therapy occurs before the step of treating the subject for the disease.

46. The method of claim 41, wherein causing the subject to undergo the therapy occurs after the step of treating the subject for the disease.

47. The method of claim 41, wherein causing the subject to undergo the therapy occurs both before and after the step of treating the subject for the disease.
